(19) United States
(12) Patent Application Publication
(10) Pub. No.: US 2017/0333904 A1
(43) Pub. Date: Nov. 23, 2017

(54) SENSOR SYSTEM AND METHOD FOR DISEASE DETECTION

(71) Applicants: Eli Lyons, Austin, TX (US); Daniel Maggs, Yokohama-shi (JP); Wojciech Bula, Tokyo (JP)

(72) Inventors: Eli Lyons, Austin, TX (US); Daniel Maggs, Yokohama-shi (JP); Wojciech Bula, Tokyo (JP)

(21) Appl. No.: 15/160,164

(22) Filed: May 20, 2016

(51) Int. Cl. B01L 3/00; G01N 21/05; C12Q 1/32

(52) U.S. Cl. CPC B01L 3/527; B01L 3/5027; C12Q 1/32; G01N 21/05; B01L 3/50; B01L 2200/04; B01L 2200/16; B01L 2300/048; B01L 2300/0627; B01L 2300/027

(57) ABSTRACT

A handheld device comprising a housing; a replaceable reagent cartridge including a plurality of liquid cartridges; a collection device, wherein the collection device is used to collect a urine sample from a user; a plurality of electrical components including a LED display, Wi-Fi connectivity, and Bluetooth; and a microfluidics platform allowing reagents from the plurality of liquid cartridges to be combined with the urine sample to create a reaction, wherein the reaction is measured with a plurality of sensors and analyzed.

### CROSS-REFERENCE TO RELATED APPLICATIONS

N/A

### BACKGROUND OF THE INVENTION

### 1. Field of the Invention

The present invention relates to a sensor system, and more particularly, the present invention relates to a sensor system and method for disease detection.

### 2. Description of Related Art

Early disease detection is critical in maintaining optimal health. Further, asymptomatic conditions and diseases may lead to significant medical costs, reduced quality of life, and lower life expectancy. Urinalysis can be an effective means of detecting such conditions and diseases. However, conventional urinalysis methods such as urine test strips are a burden for the user and make regular self-testing impractical.

Therefore, there is a need for an automated testing system that enables data collection and analysis, helping establish usual patterns and identify anomalies, while being easy to use and requiring little or no input to operate.

### BRIEF SUMMARY OF THE INVENTION

In one embodiment of the present invention a handheld device is provided comprising: a housing; a replaceable reagent cartridge including a plurality of liquid cartridges; a collection device, wherein the collection device is used to collect a urine sample from a user; a plurality of electrical components including a LED display, Wi-Fi connectivity, and Bluetooth; and a microfluidics platform allowing reagents from the plurality of liquid cartridges to be combined with the urine sample to create a reaction, wherein the reaction is measured with a plurality of sensors and analyzed.

In one embodiment, the plurality of liquid cartridges comprises a reagent A, a reagent B, a cleaning agent and/or carrier liquid, and a waste cartridge. In another embodiment, the reagent A and reagent B is beta-hydroxybutyrate dehydrogenase and nicotinamide adenine dinucleotide. In another embodiment, the replaceable reagent cartridge comprises a plurality of sockets for engagement with the plurality of liquid cartridges. In yet another embodiment, each of the plurality of liquid cartridges include a rectangular body having a top surface, a bottom surface, a reciprocal hole, a liquid chamber, and a normally closed valve. In one embodiment, each of the plurality of sockets corresponds to the reciprocal hole and when engaged the normally closed valve opens allowing liquid to flow to and from the liquid chamber

In one embodiment, the replaceable reagent cartridge further comprises a cartridge cover corresponding to each of the plurality of liquid cartridges, wherein the cover comprises a venting channel and venting hole to equalize the pressure inside the liquid cartridge. In another embodiment, the venting channel is covered with a stiff pressure-sensitive adhesive. In yet another embodiment, the microfluidics platform comprises a waste outlet and a plurality of inlets corresponding to the reagent A, the reagent B, the cleaning agent and/or carrier liquid, and the urine sample; wherein the waste outlet corresponds to the waste cartridge. In one embodiment, the microfluidics platform includes a micromixer and mandarin channel. In one embodiment, the microfluidics platform includes an optical flow cell allowing the reaction to be measured with the plurality of sensors, wherein the plurality of sensors includes a RUB and/or a LED light source and a photodetector. In another embodiment, wherein the LED display shows the results of the reaction, and the results are transferred to a computing device via Wi-Fi connectivity and/or Bluetooth and sent to Internet-based cloud computing for tracking and analysis. In yet another embodiment, the handheld device further comprises a flexible tube inlet having a filter tip and a flexible tube outlet, wherein the flexible tube inlet may be placed in a toilet for direct urine sample collection, and the flexible tube outlet may be placed in the toilet turn waste.

In another aspect of the invention, a system is provided comprising an Internet-connected computerized appliance having a processor and coupled to a data repository, the processor executing software from a non-transitory medium, the software providing an interactive interface to a disease detection software, the system enabling a user to: log on; track results generated from a handheld disease detection device, the device comprising a housing; a replaceable reagent cartridge including a plurality of liquid cartridges; a collection device, wherein the collection device is used to collect a urine sample from a user; a plurality of electrical components including a LED display, Wi-Fi connectivity, and Bluetooth; and a microfluidics platform allowing reagents from the plurality of liquid cartridges to be combined with the urine sample to create a reaction, wherein the reaction is measured with a plurality of sensors and analyzed.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a perspective view of a sensor system according to an embodiment of the present invention.

FIGS. 2A-C are perspective view of a cartridge component of the sensor system according to an embodiment of the present invention.

FIGS. 3A-C are perspective views of a liquid cartridge component of the sensor system according to an embodiment of the present invention.

FIGS. 3D-E are perspective views of valve cartridge components of the sensor system according to an embodiment of the present invention.

FIGS. 4A-B are perspective views of a cartridge cover component of the sensor system according to an embodiment of the present invention.

FIGS. 5A-H are diagrams of microfluidics platforms of the sensor system according to an embodiment of the present invention.

FIG. 6 is a diagram of a modular microfluidics platform of the sensor system according to an embodiment of the present invention.

FIG. 7 is a schematic of a sensor system according to an embodiment of the present invention,

FIG. 8 is a flowchart for a method of disease detection according to an embodiment of the present invention.

FIG. 9 is a diagram for a sensor system having an alternative collection means according to an embodiment of the present invention.

FIG. 10 is an architectural diagram of a cloud communication system according to an embodiment of the present invention.

FIG. 11 is a flowchart for a method of disease detection according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide a sensor system and method for disease detection.

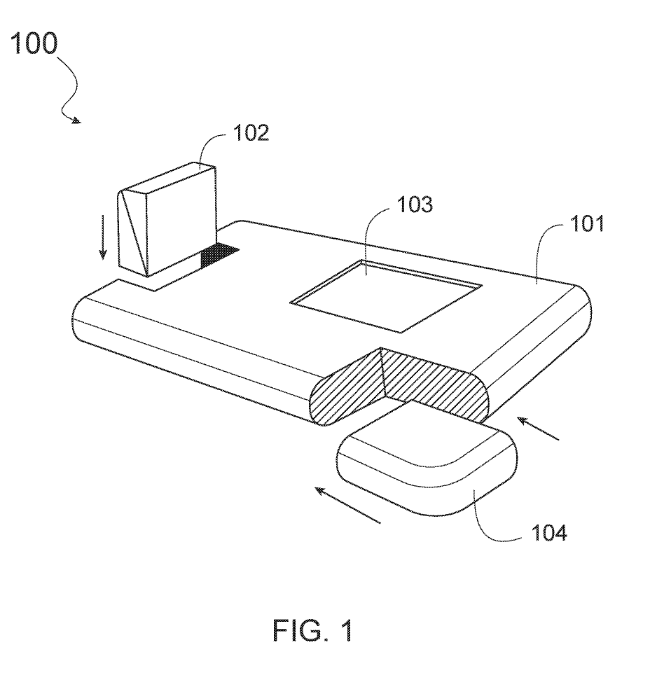

FIG. 1 is a perspective view of a sensor system 100 according to an embodiment of the present invention. Referring to FIG. 1, a sensor system 100 is illustrated, the sensor system is a handheld device providing the capability for users to collect a human liquid by-product sample, herein “urine”, for analysis and a method for disease detection. The system can test a urine sample for a range of health indicators, including but not limited to fat metabolism marker betahydroxybutyrate (BHB or Beta-hydroxybutyric acid). BHB is a type of ketone, as well known in the art. Ketones are stores of energy produced by the human body from fat when glycogen stores and blood glucose levels are insufficient to meet its energy needs. Ketones circulate in the blood and unused ketones pass into urine. It should be understood, that additional analytes may be measured by adjusting system software. This will be described in greater detail below.

100241 In one embodiment, the sensor system comprises housing 101, reagent cartridge 102, electrical components 103, and collection container 104. The housing allows the storage and connection for all components, including an internal microfluidics platform, which will be described in greater detail below. The reagent cartridge is a replaceable cartridge used to perform a range of tests by substituting different cartridges and adjusting the system software. The electrical components of the system include a LED display, Wi-Fi connectivity, and Bluetooth. The testing results are displayed on the LED display and are transferred to cloud storage for tracking and analysis. The collection container s removable and enables sample collection from either the container or directly from a stream of urine.

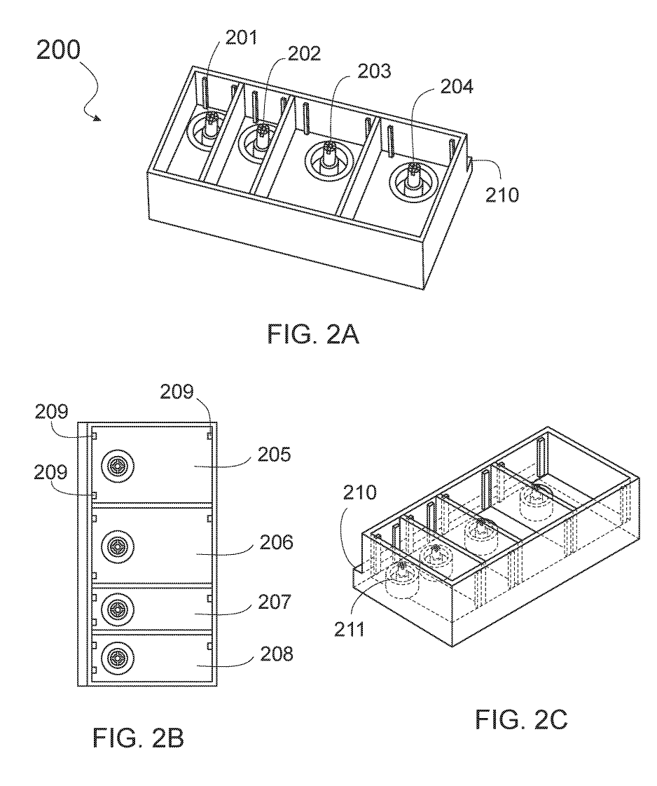

FIGS. 2A-C are perspective views of a cartridge component 200 of the sensor system according to an embodiment of the present invention. The cartridge component comprises four cartridge sockets 201 202, 203, and 204 corresponding to four receptacles 205, 206, 207 and 208 respectively. The four receptacles and corresponding cartridge sockets allow the connection of a plurality of liquid cartridges. The plurality of liquid cartridges comprises reagent A, reagent B, cleaning agent, and waste. The reagent compositions interact with the urine samples from the user to create a reaction necessary for analysis as well known in the art. Reagents A and B may be a substance, including but not limited to beta-hydroxybutyrate dehydrogenase and nicotinamide adenine dinucleotide. Examples of cleaning agents include water and detergents. A plurality of orientation pins 209 allow only appropriate liquid cartridges to be inserted in a corresponding socket 201/202/203/204. Furthermore, each liquid cartridge and its corresponding socket are marked with a specific color to aid users in inserting the correct liquid cartridge into its corresponding socket. The liquid cartridges will be discussed in greater detail below. The cartridge component further comprises a shelf 210 to assist in connection to sensor housing 101 (FIG. 1) of the sensor system.

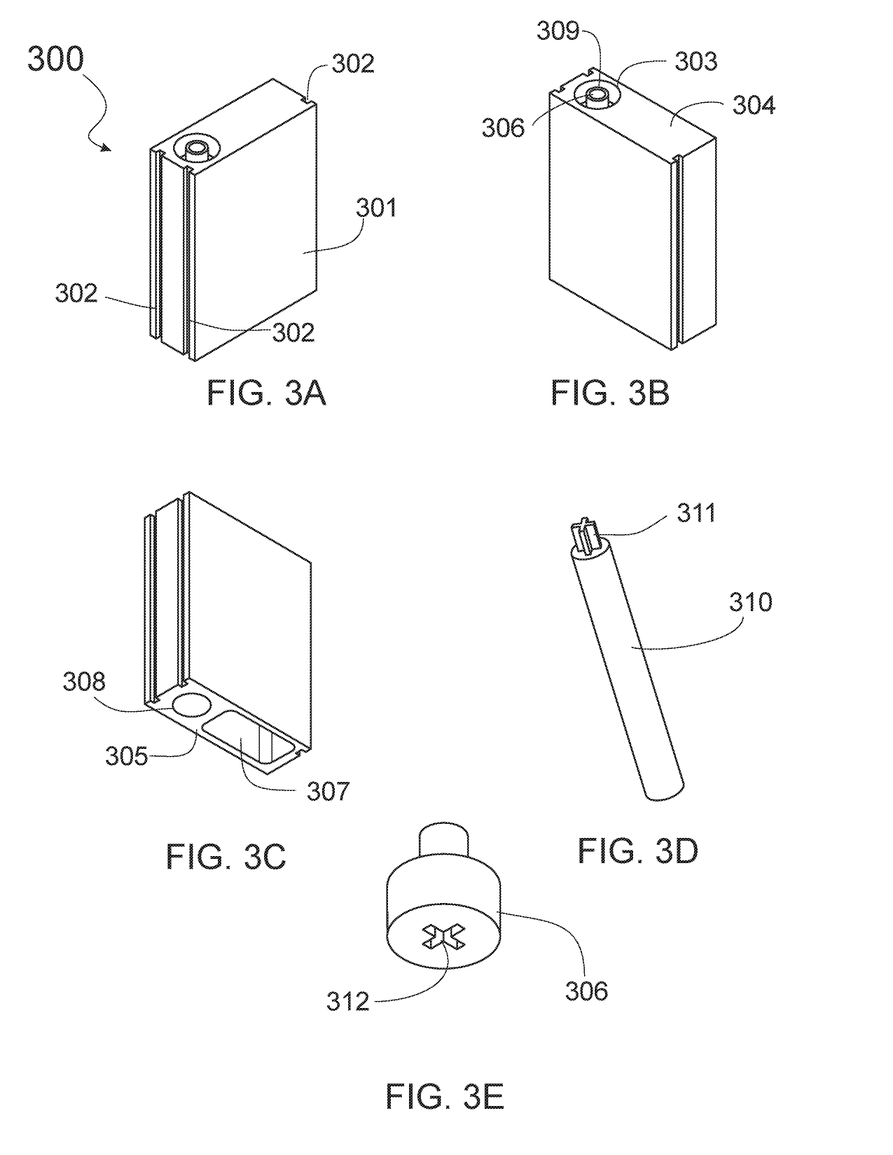

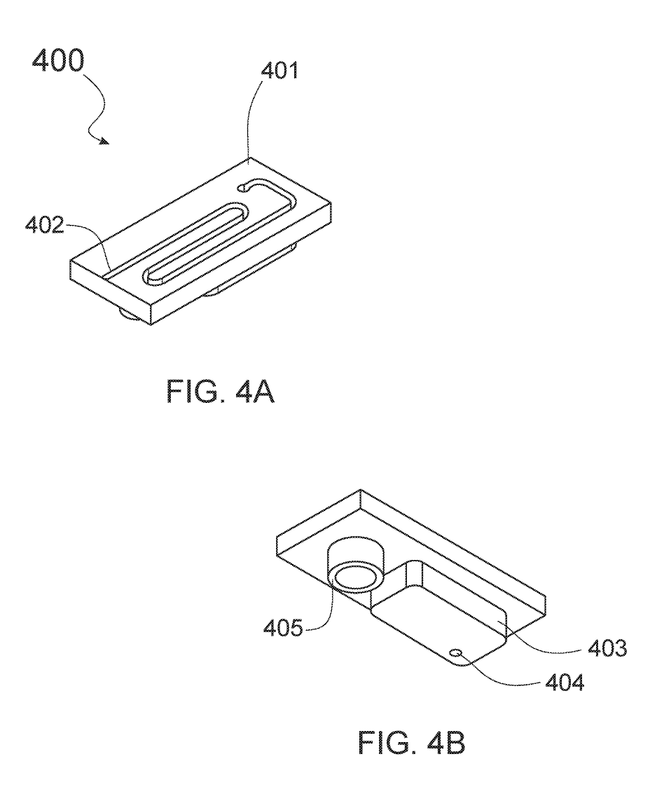

FIGS. 3A-C are perspective views of a liquid cartridge component of the sensor system according to an embodiment of the present invention. Liquid cartridge component 300 comprises a rectangular body 301 having a top surface 305, bottom surface 304, and a plurality of channels 302, wherein the plurality of channels correspond to the plurality of orientation pins illustrated in FIGS. 2A-C. It is understood that the size of the liquid cartridge is dependent on which corresponding socket 201/202/203/204 (FIGS. 2A-C) it is intended for. As previously mentioned, the liquid cartridges include a reagent A, reagent B, cleaning agent, and waste cartridge. The liquid cartridges further include a reciprocal hole 303 located on the bottom surface of the rectangular body. Each of the four cartridge sockets 201, 202, 203, and 204 (FIGS. 2A-C) are designed to enter the reciprocal hole, which lifts up a valve rod tip 306 which opens a normally closed valve allowing the liquid in the cartridge to flow from a liquid compartment 307 into a valve chamber 308 through a channel. The normally closed valve is illustrated in FIGS. 3D-E. The valve comprises rod 310 and tip 311, which corresponds to insertion 312 of valve rod tip 306. In some embodiments, the valve rod and valve rod tip are joined using any method known in the art, such as adhesives. In alternate embodiments, the valve rod and valve rod tip are a single integral unit. The valve is positioned in a normally closed position via a compression spring (not illustrated). In some embodiments, a magnet ring 309 is provided on the valve tip and an additional magnet ring 211 (FIG. 2C) is provided on each socket to secure the connection between the valve tip and the socket.

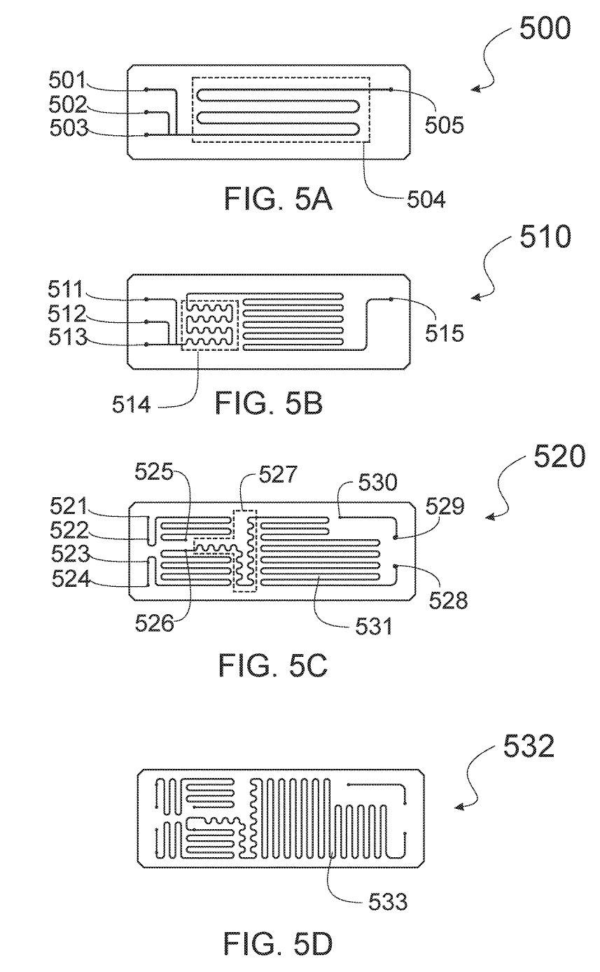

FIGS. 4A-B are perspective views of a cartridge cover component 400 of the sensor system according to an embodiment of the present invention. Referring now to FIGS. 4A-B, the cartridge cover component comprises cover tip 401, venting channel 402, seat 405, and protruding element 403 having a venting hole 404. The cartridge cover component is designed to cover the liquid cartridge, wherein the protruding element fits flush inside the liquid compartment located on the top surface of the liquid cartridge, and the seat fits inside the valve chamber (FIG. 3C). The seat holds the compression spring positioning the valve in a normally closed position as previously discussed. The venting channel and venting hold are required to equalize the pressure inside the reagent compartment. The pressure decreases when the reagent is removed from the liquid cartridge. In some embodiments, the venting channel is covered with a stiff pressure-sensitive adhesive.

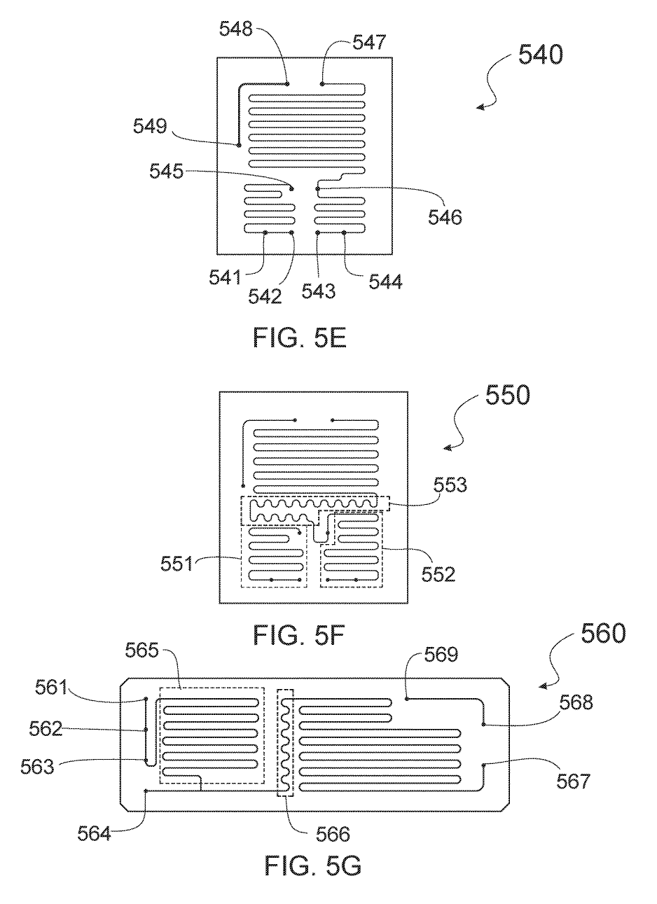

FIGS. 5A-H are diagrams of microfluidics platforms of the sensor system according to an embodiment of the present invention. Referring now to FIG. 5A, a first microfluidics platform 500 is illustrated. The first microfluidics platform comprises a first inlet 501, a second inlet 502, and a third inlet 503. The first, second, and third inlet corresponding to a reagent A, a reagent B, and a sample respectively. In some embodiments, the sample is a diluted urine sample. The reagent A, reagent B, and sample mix in the microreactor 504, as well known in the art, then exit the microfluidics platform at an outlet 505. In some embodiments, the outlet is connected to an optical flow cell for analysis.

Referring now to FIG. 5B, a second microfluidics platform 510 is illustrated. The second microfluidics platform comprises a first inlet 511, a second inlet 512, and a third inlet 513. The first, second, and third inlet corresponding to a reagent A, a reagent B, and a urine sample respectively. In some embodiments, the sample is a diluted urine sample. The reagent A, reagent B, and sample mix in a micromixer 514, as well known in the art, then exit the microfluidics platform at an outlet 515. In some embodiments, the outlet is connected to an optical flow cell for analysis.

Referring now to FIG. 5C, a third microfluidics platform 520 is illustrated. The third microfluidics platform comprises a first inlet 521, a second inlet 522, a third inlet 523, and a fourth inlet 524. The first, second, third, and fourth inlet corresponding to a reagent A, a reagent B, a urine sample, and a cleaning agent respectively. The reagent A and reagent B are mixed before entering an external valve inlet 525, wherein the pre-mixed reagent A and B exit at an external valve outlet 526. Next, the pre-mixed reagent A and B is combined with the urine sample in a micromixer 527 before entering mandarin channel 531. The cleaning agent can also enter the micromixer 527 and mandarin channel 531 before or after a reaction is performed on the microfluidics platform. Subsequently, the mixture enters an optical flow cell inlet 528 for analysis, and then exits an optical flow cell outlet 529 before proceeding to a waste outlet 530. Referring now to FIG. 5D, a fourth microfluidics platform 532 is illustrated. The fourth microfluidics platform is nearly identical to the third microfluidics platform except having a differently oriented mandarin channel 533.

Referring now to FIG. 5E, a fifth microfluidics platform 540 is illustrated. The fifth microfluidics platform comprises a first inlet 541, a second inlet 542, a third inlet 543, and a fourth inlet 544. The first, second, third, and fourth inlet corresponding to a reagent A, a reagent B, a urine sample, and a cleaning agent respectively. The reagent A and reagent B are mixed before entering an external valve inlet 545, wherein the pre-mixed reagent A and B exit at an external valve outlet 546. Next, the pre-mixed reagent A and B is combined with the urine sample and cleaning agent before entering a mandarin channel. Subsequently, the combination enters an optical flow cell inlet 527 for analysis, and then exits an optical flow cell outlet 548 before proceeding to a waste outlet 549.

Referring now to FIG. 5F, a sixth microfluidics platform 550 is illustrated. The sixth microfluidics platform is similar to the fifth microfluidics platform, however the reagent A and B are premixed in a first pre-mixing zone 551 while the urine sample and cleaning agent are premixed in a second pre-mixing zone 552 then mixed with a micromixer 553 before entering the mandarin channel.

Referring now to FIG. 5G, a seventh microfluidics platform 560 is illustrated. The seventh microfluidics platform comprises a first inlet 561, a second inlet 562, a third inlet 563, and a fourth inlet 564. The first, second, third, and fourth inlet corresponding to a reagent A, a reagent B, a urine sample, and a carrier liquid respectively. The carrier liquid is a water or buffer. The reagent A, reagent B, and urine sample are mixed in pre-mixing zone 565. The carrier liquid is injected into the platform, and the premixed reagent A, B, and urine sample are injected in-between the carrier liquid as illustrated. Next, pre-mixture and carrier liquid are mixed in a micromixer 556 before entering a mandarin channel. Subsequently, the mixture enters an optical flow cell inlet 567 for analysis, and then exits an optical flow cell outlet 568 before proceeding to a waste outlet 569. Referring now to FIG. 5H, an eighth microfluidics platform 570 is illustrated. The eighth microfluidics platform is nearly identical to the seventh microfluidics platform except having an external valve inlet 571 and external valve outlet 572, wherein the premixed reagent A, B, and urine sample are injected in-between the carrier liquid via external valve prior to the micromixer.

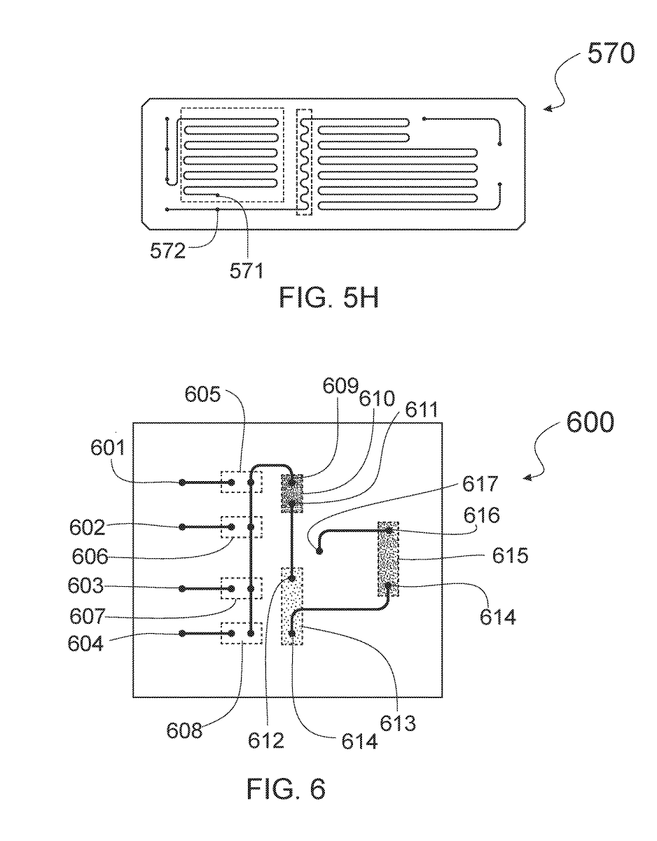

FIG. 6 is a diagram of a modular microfluidics platform 600 of the sensor system according to an embodiment of the present invention. The modular microfluidics platform comprises a first inlet 601, a second inlet, 602, a third inlet 603, and a fourth inlet 604. The first, second, third, and fourth inlet corresponding to a reagent A, a reagent 13, a urine sample, and a carrier liquid/cleaning agent respectively. Each of the four inlets are injected into the platform via microfluidic latch valves 605, 606, 607, and 608 respectively. The injected reagent A, B, urine sample, and carrier liquid/cleaning agent enter a microreactor inlet port 609, wherein the reagent A, B, urine sample, and carrier liquid/cleaning agent are mixed in a modular microreactor 610 before exiting back to the platform through a microreactor outlet port 611. The mixture then enters a micropump inlet port 612 of a micopump 613 before exiting a micropump outlet port 614. leading to an optical flow cell inlet 614. The optical flow cell inlet allowing the mixture to be analyzed via an optical flow cell 615, before exiting through an optical flow cell outlet 616 before proceeding to a waste outlet 617.

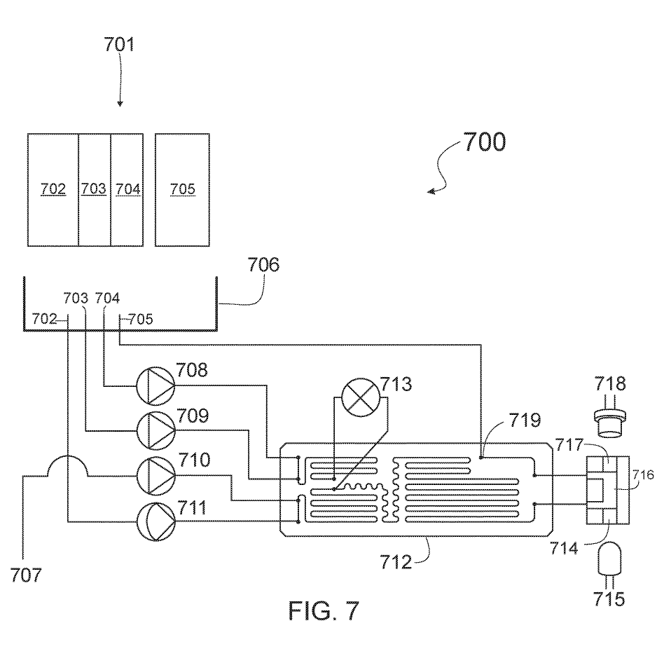

FIG. 7 is a schematic of a sensor system 700 according to an embodiment of the present invention. The schematic illustrates how the components of the system are integrated. The system comprises a cartridge 701 including four liquid cartridges housing a carrier liquid 702, reagent B 703, reagent A 704, and a waste receptacle 705. The cartridge and liquid cartridges connect to a cartridge socket 706 as previously described. A urine sample 707 is pumped into the system and more specifically a microfluidics platform 712 via a first peristaltic pump 710. Likewise, the reagent A and B are pumped into the microfluidics platform via a second and third peristaltic pump 708 and 709 respectively. Similarly, the carrier liquid is pumped into the microfluidics platform via a fourth peristaltic pump or diaphragm pump 711. It is understood that the microfluidics platform may be any of the previously disclosed microfluidics platform embodiments. In this embodiment, the reagent A and B are combined with the urine sample and carrier liquid via an external latch valve 713. After traveling through the various microfluidics platform mixers and channels as previously discussed, the combination and mixture of reagent A, B, urine sample, and carrier liquid enters a optical flow cell channel 716 having a first optically transparent window 714 adjacent to a RGB or LED light source and a second optically transparent window 717 adjacent to a photodetector 718. The optical flow cell is where reagent activity is measured via the sensors. In some embodiments, the urine sample and reagent mixture pass in-between an LED and an optical sensor and the color absorption is measured. In some embodiments, fluorescent reagents are present and the LED is used to excite the fluorescent reagents. The reagent activity is measured by a camera or other light-sensitive sensor. Finally, after analysis the mixtures exit the optical flow cell channel proceeding to a waste outlet 619 which connects to the waste liquid cartridge as illustrated.

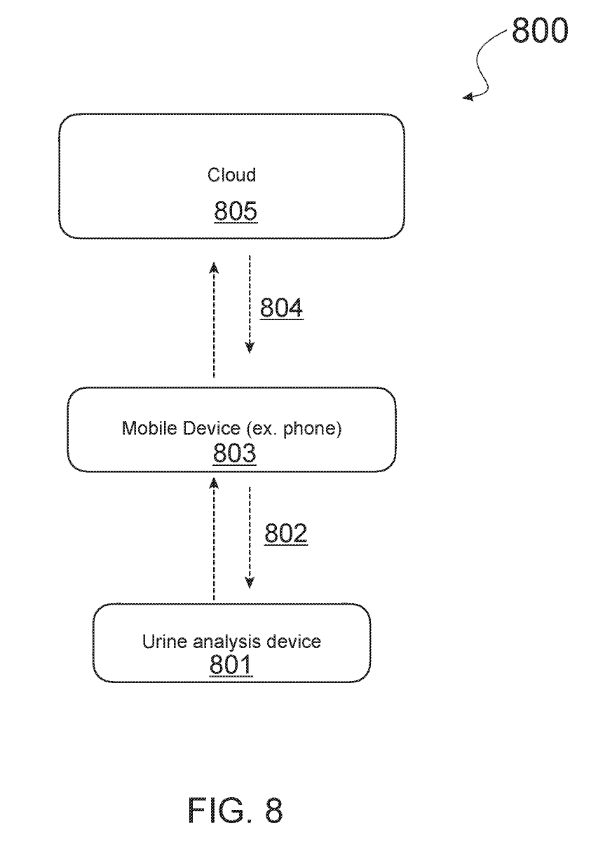

FIG. 8 is a flowchart for a method of disease detection 800 according to an embodiment of the present invention. The method of disease detection comprises urine analysis device or sensor system 801 in communication 802 with a computing device or mobile device such as a smartphone 803. The computing device comprising a processor, storage, and memory as known in the art. For instance, the analysis performed on the device is sent via Bluetooth to a user's smartphone. Next, the analysis is transferred to the cloud 805 via an Internet network system 804 for complete analysis and results.

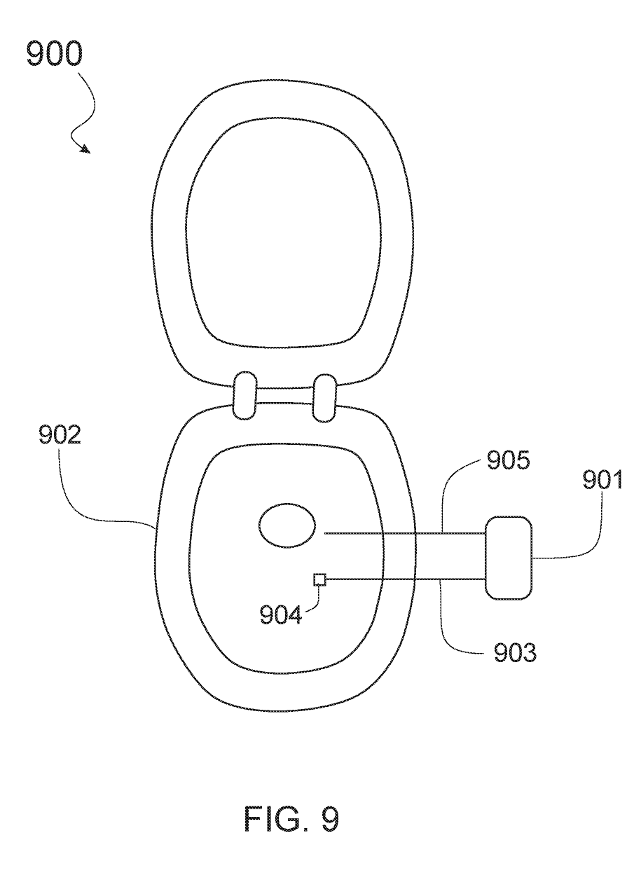

FIG. 9 is a diagram 900 for a sensor system 901 having an alternative collection means according to an embodiment of the present invention. Referring now to FIG. 9, the sensor system is attached or located in proximity to a toilet 902, wherein a flexible tube 304 with a filter 904 is placed in the toilet bowl and connected to the sensor system allowing the collection of a urine sample via directly from the toilet providing a more convenient and sanitary method of collected the urine sample in relation to the embodiments described above. After the sensor system analyses the sample according to the methods described above, all the waste is returned to the toilet via flexible tube 905.

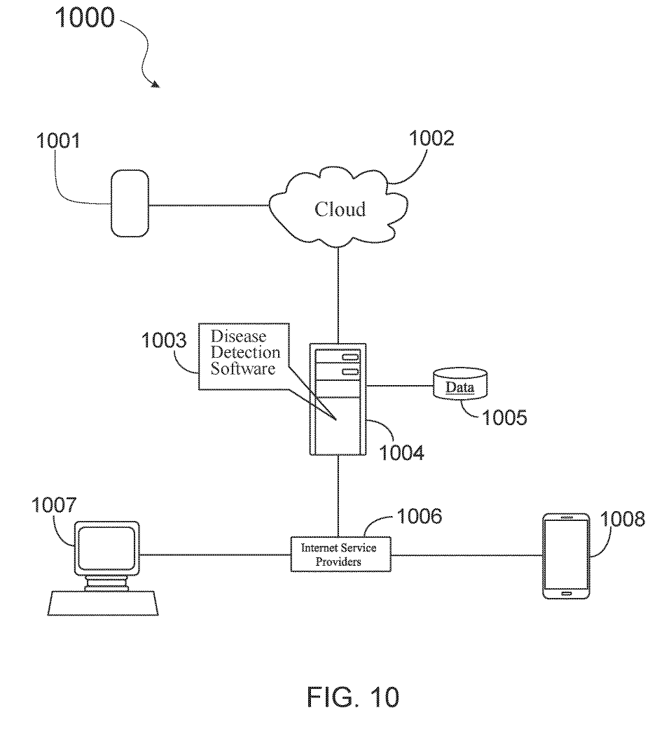

FIG. 10 is an architectural diagram of a cloud communication system 1000 according to an embodiment of the present invention. The Internet-connected system comprises one or more Internet-connected server 1004 executing disease detection software 1003 from non-transitory media. Server 1004 is connected to a data repository 1005, which may be any sort of data storage known in the art. A user on a computerized device 1007 or mobile device 1008 is connected to the Internet-connected server via an Internet service provider (ISP) 1006, allowing the users to access the system. As described in FIG. 8, analysis preformed on sensor system device 1001 is transferred via Bluetooth or other wireless technology to the computerized or mobile device, which connects to the cloud 1002 allowing the user to access the software for complete analysis, tracking, and results. The analysis and results are provided on the user's computerized device.

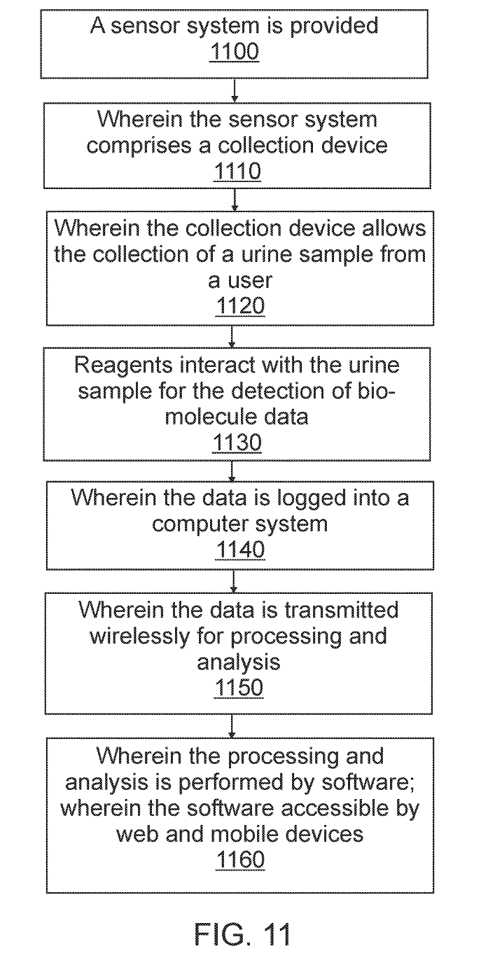

FIG. 11 is a flowchart for a method of disease detection according to an embodiment of the present invention. In operation 1100 a sensor system is provided, wherein the sensor system comprises a collection device 1110. The collection device allows the collection of a urine sample from a user 1120. In operation 1130, reagents interact with the urine sample for the detection of bio-molecule data. In operation 1140, the data is logged into a computer system. Next in operation 1150, the data is transmitted wirelessly for processing and analysis. Last in operation 1160, the processing and analysis is performed by software, wherein the software is accessible by web and mobile devices.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. For instance, in some embodiments, the system can be adapted to perform other analysis by swapping software and/or sensor or measurement devices.

In one embodiment, a measurement device may include a pH sensor which detects the urine pH value. The pH of urine is a measure of its hydrogen ion concentration. A pH below 7.0 indicates acid urine. A pH above 7.0 indicates alkaline urine. For example, for chronic dehydration, one symptom is a detection of urine pH below 6.0 over an extended period of time. In another embodiment, the measurement device may include an electrochemical measurement chip. In this particular embodiment, urine collected interacts with the electrochemical measurement chip, in which reagents specific to bio-molecules in urine detect a change in voltage potential due to interaction between the reagents and bio-molecules, such as albumin and prostate specific antigen (PSA) in the urine. In other embodiments, the system can perform a comprehensive health monitoring solution detects a wide range of conditions, including cancers, infections, inflammations and STDs.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A handheld device comprising:
a housing;
a replaceable reagent cartridge including a plurality of liquid cartridges;
a collection device, wherein the collection device is used to collect a urine sample from a user;
a plurality of electrical components including a LED display, Wi-Fi connectivity, and Bluetooth; and
a microfluidics platform allowing reagents from the plurality of liquid cartridges to be combined with the urine sample to create a reaction, wherein the reaction is measured with a plurality of sensors and analyzed.

2. The handheld device of claim 1, wherein the plurality of liquid cartridges comprises a reagent A, a reagent B, a cleaning agent and/or carrier liquid, and a waste cartridge.

3. The handheld device of claim 2, wherein the reagent A and reagent B is beta-hydroxybutyrate dehydrogenase and nicotinamide adenine dinucleotide.

4. The handheld device of claim 1, wherein the replaceable reagent cartridge comprises a plurality of sockets for engagement with the plurality of liquid cartridges.

5. The handheld device of claim 4, wherein each of the plurality of liquid cartridges include a rectangular body having a top surface, a bottom surface, a reciprocal hole, a liquid chamber, and a normally closed valve.

6. The handheld device of claim 5, wherein each of the plurality of sockets corresponds to the reciprocal hole and when engaged the normally closed valve opens allowing liquid to flow to and from the liquid chamber.

7. The handheld device of claim 6, wherein the replaceable reagent cartridge further comprises a cartridge cover corresponding to each of the plurality of liquid cartridges, wherein the cover comprises a venting channel and venting hole to equalize the pressure inside the liquid cartridge.

8. The handheld device of claim 7, wherein the venting channel is covered with a stiff pressure-sensitive adhesive.

9. The handheld device of claim 2, wherein the microfluidics platform comprises a waste outlet and a plurality of inlets corresponding to the reagent A, the reagent B, the cleaning agent and/or carrier liquid, and the urine sample; wherein the waste outlet corresponds to the waste cartridge.

10. The handheld device of claim 9, wherein the microfluidics platform includes a micromixer and mandarin channel.

11. The handheld device of claim 10, wherein the microfluidics platform includes an optical flow cell allowing the reaction to be measured with the plurality of sensors, wherein the plurality of sensors include a RGB and/or a LED light source and a photodetector.

12. The handheld device of claim 1, wherein the LED display shows the results of the reaction, and the results are transferred to a computing device via Wi-Fi connectivity and/or Bluetooth and sent to Internet-based cloud computing for tracking and analysis.

13. The handheld device of claim 1, wherein the handheld device further comprises a flexible tube inlet having a filter tip and a flexible tube outlet, wherein the flexible tube inlet may be placed in a toilet for direct urine sample collection, and the flexible tube outlet may be placed in the toilet to return waste.

14. A system comprising:
an Internet-connected computerized appliance having a processor and coupled to a data repository, the processor executing software from a non-transitory medium, the software providing an interactive interface to a disease detection software, the system enabling a user to: log on;
track results generated from a handheld disease detection device, the device comprising a housing; a replaceable reagent cartridge including a plurality of liquid cartridges; a collection device, wherein the collection device is used to collect a urine sample from a user; a plurality of electrical components including a LED display, Wi-Fi connectivity, and Bluetooth; and a microfluidics platform allowing reagents from the plurality of liquid cartridges to be combined with the urine sample to create a reaction, wherein the reaction is measured with a plurality of sensors and analyzed.
